SEQUENCE LISTING (15 sequences)

SEQ ID NO: 1 - DNA, 27 bp, synthetic construct
  source 1..27: mol_type = other DNA
tgagcggataacaattcccctctagaa

SEQ ID NO: 2 - DNA, 26 bp, synthetic construct
  source 1..26: mol_type = other DNA
agcaggttcgcggactgggacggatc

SEQ ID NO: 3 - DNA, 26 bp, synthetic construct
  source 1..26: mol_type = other DNA
gatccgtcccagtccgcgaacctgct

SEQ ID NO: 4 - DNA, 29 bp, synthetic construct
  source 1..29: mol_type = other DNA
aataaattagcactttgactagggtcgtc

SEQ ID NO: 5 - DNA, 29 bp, synthetic construct
  source 1..29: mol_type = other DNA
gacgaccctagtcaaagtgctaatttatt

SEQ ID NO: 6 - DNA, 37 bp, synthetic construct
  source 1..37: mol_type = other DNA
ccgccgccggatcctttcgcgtcgaccttaggagctt

SEQ ID NO: 7 - DNA, 235 bp, synthetic construct
  source 1..235: mol_type = other DNA
tgagcggataacaattcccctctagaaataattttgtttaactttaagaaggagatataccaatgcaccaccaccatcaccacgtggacgcgaaattcgacgcggataacaactttaacaaagagcagcagaacgcattctatgaaatcctgaacatgccgaacctgaatgaagaacagctgaacggcttcatccagtctctgaaagatgatccgtcccagtccgcgaacctgct

SEQ ID NO: 8 - DNA, 230 bp, synthetic construct
  source 1..230: mol_type = other DNA
gatccgtcccagtccgcgaacctgctgagcgaagctaaaaaactgaacgaaagccaggcgccgaagcaggcgccgaaagttgatgctaagtttgacgcagacaataatttcaataaggaacaacaaaatgcgttttacgagattcttaatatgccaaatcttaacgaggagcaacttaacggttttattcaaagcctaaaagacgaccctagtcaaagtgctaatttatt

SEQ ID NO: 9 - DNA, 93 bp, synthetic construct
  source 1..93: mol_type = other DNA
gacgaccctagtcaaagtgctaatttattatctgaggcgaagaagttaaatgagagtcaagcacctaaacaagctcctaaggtcgacgcgaaa

SEQ ID NO: 10 - DNA, 471 bp, synthetic construct
  source 1..471: mol_type = other DNA
ctagaaataattttgtttaactttaagaaggagatataccaatgcaccaccaccatcaccacgtggacgcgaaattcgacgcggataacaactttaacaaagagcagcagaacgcattctatgaaatcctgaacatgccgaacctgaatgaagaacagctgaacggcttcatccagtctctgaaagatgatccgtcccagtccgcgaacctgctgagcgaagctaaaaaactgaacgaaagccaggcgccgaagcaggcgccgaaagttgatgctaagtttgacgcagacaataatttcaataaggaacaacaaaatgcgttttacgagattcttaatatgccaaatcttaacgaggagcaacttaacggttttattcaaagcctaaaagacgaccctagtcaaagtgctaatttattatctgaggcgaagaagttaaatgagagtcaagcacctaaacaagctcctaagg

SEQ ID NO: 11 - DNA, 5226 bp, synthetic construct
  source 1..5226: mol_type = other DNA
tcgacgcgaaaggatccggcggcggctgctaactcgaggcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccct

SEQ ID NO: 12 - DNA, 37 bp, synthetic construct
  source 1..37: mol_type = other DNA
aatgcaccaccaccatcaccacgtcgacgcgaaattc

SEQ ID NO: 13 - DNA, 39 bp, synthetic construct
  source 1..39: mol_type = other DNA
gagttagcagccgccgccggatcccttaggagcttgttt

SEQ ID NO: 14 - protein, 284 aa, synthetic construct
HHHHHHVDAKFDADNNFNKEQQNAFYEILNMPNLNEEQLNGFIQSLKDDPSQSANLLSEAKKLNESQAPKQAPKVDAKFDADNNFNKEQQNAFYEILNMPNLNEEQLNGFIQSLKDDPSQSANLLSEAKKLNESQAPKQAPKVDAKFDADNNFNKEQQNAFYEILNMPNLNEEQLNGFIQSLKDDPSQSANLLSEAKKLNESQAPKQAPKVDAKFDADNNFNKEQQNAFYEILNMPNLNEEQLNGFIQSLKDDPSQSANLLSEAKKLNESQAPKQAPKGSGGGC

SEQ ID NO: 15 - protein, 420 aa, synthetic construct
HHHHHHVDAKFDADNNFNKEQQNAFYEILNMPNLNEEQLNGFIQSLKDDPSQSANLLSEAKKLNESQAPKQAPKVDAKFDADNNFNKEQQNAFYEILNMPNLNEEQLNGFIQSLKDDPSQSANLLSEAKKLNESQAPKQAPKVDAKFDADNNFNKEQQNAFYEILNMPNLNEEQLNGFIQSLKDDPSQSANLLSEAKKLNESQAPKQAPKVDAKFDADNNFNKEQQNAFYEILNMPNLNEEQLNGFIQSLKDDPSQSANLLSEAKKLNESQAPKQAPKVDAKFDADNNFNKEQQNAFYEILNMPNLNEEQLNGFIQSLKDDPSQSANLLSEAKKLNESQAPKQAPKVDAKFDADNNFNKEQQNAFYEILNMPNLNEEQLNGFIQSLKDDPSQSANLLSEAKKLNESQAPKQAPKGSGGGC

(19) United States
(12) Patent Application Publication
(10) Pub. No.: US 2025/0042953 A1
(43) Pub. Date: Feb. 6, 2025

(54) ALKALI-TOLERANT MUTATION METHOD OF PROTEIN A

(71) Applicant: Hangzhou NeuroPeptide Biological Science and Technology Incorporation, Ltd. ( NUPTEC), Hangzhou (CN)

(72) Inventors: Zhenfei Gao, Hangzhou (CN); Yongchang Qian, Hangzhou (CN); Ke Zhang, Hangzhou (CN); Cangang Lai, Hangzhou (CN)

(21) Appl. No.: 18/499,176

(22) Filed: Oct. 31, 2023

(30) Foreign Application Priority Data
Jul. 31, 2023 (CN) 202310950502.X

(51) Int. Cl. C07K 14/31; C12N 1/20; C12N 5/00; C12N 15/70; C12Q 1/686; C12Q 1/6876; G01N 33/543

(52) U.S. Cl. CPC C07K 14/31; C12N 1/205; C12N 5/0018; C12N 15/70; C12Q 1/686; C12Q 1/6876; G01N 33/543; C12N 2500/34; C12R 2001/19

(57) ABSTRACT

An alkali-tolerant mutation method of a protein A includes: adding a GGGC sequence to a C-terminus of the protein A, and selecting multiple mutation sites of an A domain of the protein A; constructing multiple vectors on the multiple mutation sites of the protein A based on the domain A; expressing multiple mutant proteins by using the multiple vectors at the multiple mutation sites of the protein A, respectively; detecting expression results of the multiple mutant proteins to obtain detection results; and screening an optimal mutant protein of the protein A based on the detection results.

### TECHNICAL FIELD

The disclosure relates to the technical field of biological engineering, particularly to an alkali-tolerant mutation method of a protein A.

### STATEMENT REGARDING SEQUENCE LISTING

The sequence listing associated with this application is provided in text format in lieu of a paper copy and is hereby incorporated by reference into the specification. The name of the XML file containing the sequence listing is 23016TZRH-USP1-SL.xml. The XML file is 21,172 bytes; is created on Oct. 23, 2023; and is being submitted electronically via EFS-Web.

### BACKGROUND

During producing protein molecules, it is necessary to remove pollutants to ensure a purity of a protein product. The above mentioned pollutants include non-target biomolecules or non-target microorganisms such as proteins, carbohydrates, lipids, bacteria, and viruses. These pollutants are usually removed from a substrate after the target product is eluted, which is convenient to regenerate a substrate before a subsequent application. A method for removing the pollutants usually includes a process known as cleaning in place (CIP), in which a reagent capable of eluting the pollutants from a stationary phase is used. At present, sodium hydroxide (NaOH) is the most widely used reagent, and a concentration of the NaOH can vary from 0.1 moles per liter (M) to 1 M depending on pollution degree and substrate property. NaOH is an effective CIP reagent that can reduce the pollutants such as microorganisms, proteins, lipids and nucleic acids in many ways. Another advantage of NaOH is that it can be easily removed without any further treatment. However, the method described herein needs to expose the substrate to an extremely alkaline condition with a potential of hydrogen (pH) exceeding 13, which requires that the substrate has a high alkali tolerance.

### SUMMARY

In order to solve the technical problems mentioned in the background above, some embodiments of the disclosure provide an alkali-tolerant mutation method of a protein A, which includes the following steps:
adding a GGGC sequence to a C-terminus of the protein A, and selecting multiple mutation sites of an A domain of the protein A;
constructing multiple vectors on the multiple mutation sites of the protein A based on the domain A;
expressing multiple mutant proteins by using the multiple vectors at the multiple mutation sites of the protein A, respectively;
detecting expression results of the multiple mutant proteins to obtain detection results; and
screening an optimal mutant protein of the protein A based on the detection results.

In an embodiment, the constructing multiple vectors on the multiple mutation sites of the protein A based on the domain A includes the following steps:
step 1, obtaining multiple design primers, including: a first design primer, a second design primer, . . . , and a n-th design primer;
step 2, introducing two of the multiple design primers into mutation sites of a template based on a polymerase chain reaction (PCR) to obtain a mutated target gene;
step 3, performing double digestion on the template and the mutated target gene to obtain a first enzymatic cleavage product and a second enzymatic cleavage product, respectively;
step 4, performing a T4 ligation between the first enzymatic cleavage product and the second enzymatic cleavage product to obtain a ligation product, culturing the ligation product on a Lysogeny broth (LB) agar plate with kanamycin to obtain a plasmid, and then extracting the plasmid on the Lysogeny broth (LB) agar plate with kanamycin; and
step 5, using the plasmid as another template, introducing remaining design primers of the multiple design primers into mutation sites of the plasmid to repeat the step 3 and the step 4, thereby constructing the multiple vectors on the multiple mutation sites of the protein A based on the domain A.

In an embodiment, the constructing multiple vectors on the multiple mutation sites of the protein A based on the domain A further includes the following steps:
step 6, using the multiple vectors constructed on the multiple mutation sites of the protein A based on the domain A as still another templates, introducing the remaining design primers of the multiple design primers into mutation sites of the still another templates to obtain mutated target genes corresponding to the still another templates; and
repeating the step 3 to the step 5 with the still another templates and the mutated target genes corresponding to the still another templates to obtain multiple reconstructed vectors as the multiple vectors constructed on the multiple mutation sites of the protein A based on the domain A.

In an embodiment, after the performing double digestion on the template and the mutated target gene to obtain a first enzymatic cleavage product and a second enzymatic cleavage product, respectively, of the step 3, the method includes:

adding 10× loading buffer to the first enzymatic cleavage product to conduct a 1% agarose gel electrophoresis to obtain a gel, then observing a nucleic acid band, and cutting and recycling the gel.

In an embodiment, the culturing the ligation product on a LB agar plate with kanamycin to obtain a plasmid, and then extracting the plasmid from the LB agar plate with kanamycin in the step 4 includes the following steps:
transferring the ligation product to DH5α competent cells, incubating the transferred DH5α competent cells on ice, and then performing a heat shock;
re-incubating the incubated DH5α competent cells on ice;
adding a non-resistant LB medium to the re-incubated DH5α competent cells in a flask to perform shake-flask culture; centrifuging the flask to take a resuspended bacterial solution with the non-resistant LB medium, and smearing the resuspended bacterial solution on the LB agar plate with kanamycin;
placing the LB agar plate with kanamycin in an incubator for cultivation; and
after a period of time, screening positive monoclonal bacterial strains P on the LB agar plate with kanamycin to perform sample sequencing to obtain sequencing results, taking a bacterial strain with a complete sequencing result based on the sequencing results, and extracting the plasmid from the bacterial strain with the complete sequencing result.

In an embodiment, the expressing multiple mutant proteins by using the multiple vectors at the multiple mutation sites of the protein A, respectively, includes the following steps:
transferring the multiple vectors into multiple BL21 competent cells, respectively, to mix the multiple vectors with the multiple BL21 competent cells evenly to obtain multiple mixtures; incubating the multiple mixtures on ice and then performing a heat shock, then re-incubating the multiple incubated mixtures on ice; adding non-resistant LB media to the multiple re-incubated mixtures in flasks to perform shake-flask culture; centrifuging the flasks to take resuspended bacterial solutions with the non-resistant LB media, and smearing the resuspended bacterial solutions on multiple LB agar plates with kanamycin; placing the multiple LB agar plates with kanamycin in incubators for cultivation;
after a period of time, cultivating positive monoclonal bacterial strains taken from the multiple LB agar plates with kanamycin, and then adding 0.1% isopropyl β-D-thiogalactopyranoside (IPTG) to the positive monoclonal bacterial strains, respectively, for a continue cultivation for a period of time to obtain multiple culture products;
performing a sodium dodecyl-sulfate polyacrylamide gel electrophoresis (SDS-PAGE) on the multiple culture products, respectively, to obtain multiple expressed culture products; and
transferring the multiple expressed culture products to multiple LB media, respectively, to cultivate for a period of time, then respectively adding 0.1% IPTG to the multiple LB media for continue cultivation for a period of time and collecting multiple expressed bacterial proteins.

In an embodiment, the detecting expression results of the multiple mutant proteins to obtain detection results includes the following steps:
placing each of the multiple expressed bacterial proteins in an alkaline environment for a treatment;
performing an activity detection for each of the expressed bacterial proteins placed in the alkaline environment by a double immunodiffusion test; and
obtaining data of the activity detection.

In an embodiment, the screening an optimal mutant protein of the protein A based on the detection results includes:

selecting an alkali-tolerant mutant protein A with a highest activity after a preset time as the optimal mutant protein A based on the data of the activity detection.

### BRIEF DESCRIPTION OF DRAWINGS

Attached drawings included in the disclosure are used to provide a further understanding of the disclosure, making other features, objectives, and advantages of the disclosure more apparent. The illustrative embodiments, the attached drawings, and their corresponding descriptions in the disclosure are used to explain the disclosure and do not constitute improper limitations of the disclosure.

In addition, throughout the attached drawings, identical or similar reference numerals represent identical or similar elements. It should be understood that the attached drawings are illustrative, and components and elements may not necessarily be drawn to scale.

FIG. 1 illustrates an overall flowchart of an alkali-tolerant mutation method of a protein A according to an embodiment of the disclosure.

FIG. 2 illustrates a histogram of 24-hour protein activity changes of a staphylococcal protein A 5-6 (SpA5-6) and a comparative r-SpA that are treated with 1 mole per liter (M) sodium hydroxide (NaOH).

FIG. 3 illustrates results of double digestion with restriction endonucleases <Xba I, Sal I> of a PCR product of an overlap PCR; where line 1 represents a gel running result of the double digestion, and line 2 represents a marker.

FIG. 4 illustrates results of double digestion with restriction endonucleases <Sal I, BamH I> on a vector and a target gene with a single nucleotide mutation; where line 1 represents a gel running result of the double digestion with the restriction endonucleases <Sal I, BamH I> of the vector, line 2 represents a marker, and line 3 represents a gel running result of the double digestion with the restriction endonucleases <Sal I, BamH I> of the target gene with the single nucleotide mutation.

FIG. 5 illustrates a PCR result by using a domain A-5 as a template; where line 1 represents the PCR result and line 2 represents a marker.

FIG. 6 illustrates a protein gel chart of the SpA5-6; where line 1 represents a marker and line 2 represents the SpA5-6.

FIG. 7 illustrates a protein gel chart of a SpA5; where line 1 represents a marker and line 2 represents the SpA5.

FIG. 8 illustrates a histogram of 24-hour protein activity changes of the SpA5 and the comparative r-SpA that are treated with 1 M NaOH.

### DETAILED DESCRIPTION OF EMBODIMENTS

Embodiments of the disclosure will be described in more detail below with reference to the attached drawings. Although some illustrated embodiments of the disclosure are shown in the attached drawings, it should be understood that the disclosure can be implemented in various forms and should not be limited to the embodiments described here. On the contrary, the embodiments of the disclosure are used to facilitate understanding the disclosure more thoroughly and completely. It should be understood that the attached drawings and the embodiments disclosed in the disclosure are only for illustrative purposes and are not intended to limit the scope of the protection of the disclosure.

Furthermore, it should be noted that for the convenience of description, the attached drawings only illustrate parts related to the disclosure. Without conflict, the embodiments and features in the embodiments disclosed in the disclosure can be combined with each other.

It should be noted that concepts such as “first” and “second” mentioned in the disclosure are only used to distinguish different devices, modules, or units, and are not intended to limit an order or an interdependence of functions performed by these devices, modules, or units.

It should be noted that modifications such as “one” and “multiple” mentioned in the disclosure are illustrative rather than restrictive, and those skilled in the related art should understand that unless otherwise explicitly stated in the context, they should be understood as “one or more”.

The disclosure is described in detail with reference to the attached drawings combined with the embodiments.

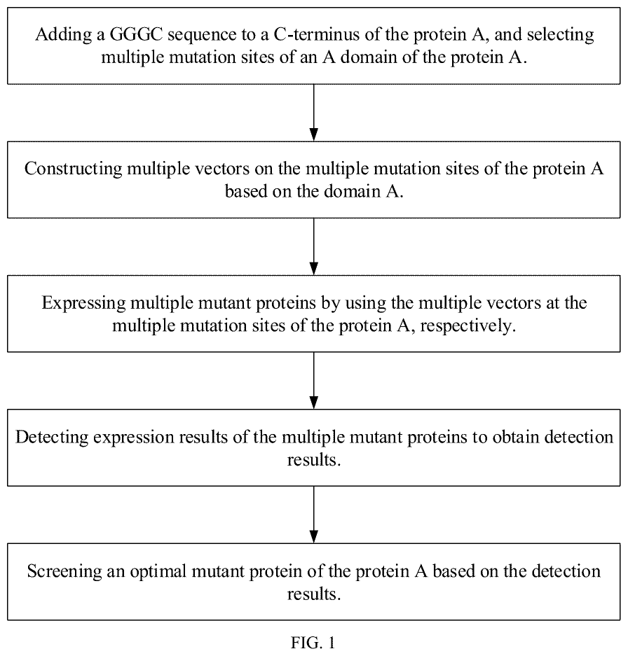

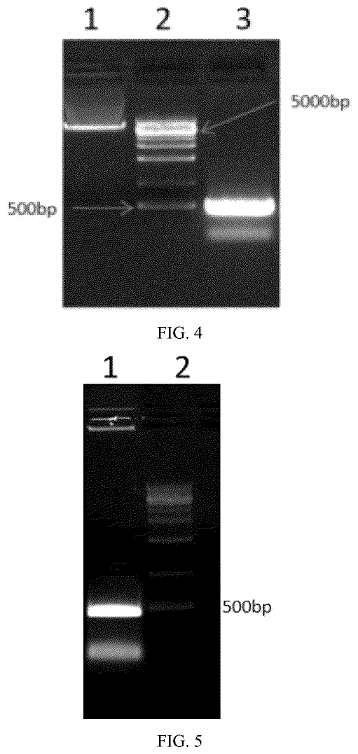

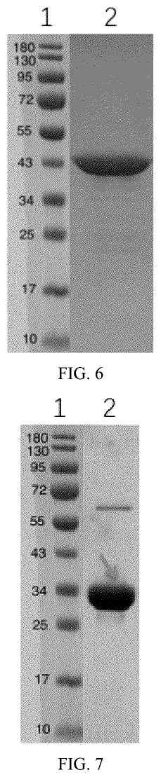

With reference to FIGS. 1-8, the disclosure provides an alkali-tolerant mutation method of a protein A, including the following steps:
adding a GGGC sequence to a C-terminus of the protein A, and selecting multiple mutation sites of an A domain of the protein A;
constructing multiple vectors on the multiple mutation sites of the protein A based on the domain A;
expressing multiple mutant proteins by using the multiple vectors at the multiple mutation sites of the protein A, respectively;
detecting expression results of the multiple mutant proteins to obtain detection results; and
screening an optimal mutant protein of the protein A based on the detection results.

In an illustrated embodiment, the constructing multiple vectors on the multiple mutation sites of the protein A based on the domain A includes the following steps:
step 1, obtaining multiple design primers, including: a first design primer, a second design primer, . . . , and a n-th design primer;
step 2, introducing two of the multiple design primers into mutation sites of a template based on a polymerase chain reaction (PCR) to obtain a mutated target gene;
step 3, performing double digestion on the template and the mutated target gene to obtain a first enzymatic cleavage product and a second enzymatic cleavage product, respectively;
step 4, performing a T4 ligation between the first enzymatic cleavage product and the second enzymatic cleavage product to obtain a ligation product, culturing the ligation product on a Lysogeny broth (LB) agar plate with kanamycin to obtain a plasmid, and then extracting the plasmid on the LB agar plate with kanamycin; and step 5, using the plasmid as another template, introducing remaining design primers of the multiple design primers into mutation sites of the plasmid to repeat the step 3 and the step 4, thereby constructing the multiple vectors on the multiple mutation sites of the protein A based on the domain A.

Specially, in the step 1, the multiple design primers include the first design primer, the second design primer, . . . , and the n-th design primer. In an illustrated embodiment, the disclosure provides a method for preparing a vector as follows:
when preparing the vector, the multiple design primers are used in the vector, i.e., a first design primer, a second design primer, a third design primer, a fourth design primer, a fifth design primer, and a sixth design primer are designed. Furthermore, the first design primer, the second design primer, the third design primer, the fourth design primer, the fifth design primer, and the sixth design primer are respectively domainA F1′, domainA R1′, domainA F2′, domainA R2′, domainA F3′, and domainA R3′:
the gene sequence of the domainA F1′ is SEQ ID NO: 1 as TGAGCGGATAACAATTCCCCTCTAGAA;
the gene sequence of the domainA R1′ is SEQ ID NO: 2 as AGCAGGTTCGCGGACTGGGACGGATC;
the gene sequence of the domainA F2′ is SEQ ID NO: 3 as GATCCGTCCCAGTCCGCGAACCTGCT;
the gene sequence of the domainA R2′ is SEQ ID NO: 4 as AATAAATTAGCACTTTGACTAGGGTCGTC;
the gene sequence of the domainA F3′ is SEQ ID NO: 5 as GACGACCCTAGTCAAAGTGCTAATTTATT; and
the gene sequence of the domainA R3′ is SEQ ID NO: 6 as CCGCCGCCGGATCCTTTCGCGTCGACCTTAGGAGCTT.

Specially, in the step 2, two of the multiple design primers are introduced into the mutation sites of the template based on the PCR, thereby to obtain the mutated target gene; the template is a single repeat Staphylococcus protein A member 11 (SpA11) protein expression precursor vector in domain A; and the mutation sites of the template include: R27L (also referred to p.R27L, substitution-missense at position 27 with R-L), N28L (referred to substitution-missense at position 28 with N-L), K35Y (referred to substitution-missense at position 35 with K-Y), and S41W (referred to substitution-missense at position 41 with S-W).

The PCR includes a PCR system and a PCR program.

In an illustrated embodiment, the PCR system is as follows:

| Reagent name | Usage amount |
|---|---|
| PrimeSTAR ® Max | 20 μL |
| F′ | 2 μL |
| R′ | 2 μL |
| 6#plasmid | 0.5 μL |
| H₂O | 15.5 μL |

In an illustrated embodiment, the PCR program is as follows:

after preparing the required reagents for the PCR program, a PCR instrument is preheated at 98 degrees Celsius (° C.) for 3 minutes (min) to denature deoxyribonucleic acid (DNA) of the PCR template (also referred as to the 6 #plasmid), and then an amplification cycle is performed, including: maintaining at 98° C. for 30 seconds(s) to denature the PCR template; decreasing the temperature to a renaturation temperature at 50° C. and maintaining for 30 s to fully anneal designated primers (F′ including: the first design primer F1′, the third design primer F2′, and the fifth design primer F3′ and R′ including: the second design primer R1′, the fourth design primer R2′, and the sixth design primer R3′) and the PCR template; increasing the temperature to 72° C. and maintaining for 3 s for amplification, thereby extending the designated primers on the PCR template and synthesizing DNA; thereafter the above-mentioned cycle is repeated for 32 times to obtain a PCR product, and then the temperature is maintained at 72° C. for 5 min to extend the PCR product completely; and finally, the PCR product is stored at 4° C.

Then, 10× loading buffer is added to the PCR product to conduct a 1% agarose gel electrophoresis at 150 volts (V) for 15 min to obtain a gel, thereafter observing a nucleic acid band and cutting and recycling the gel (also referred as to the recovered PCR product).

Then, the recovered PCR product is performed an overlap PCR. And the overlap PCR system used herein is as follows:

| Reagent name | Usage amount |
|---|---|
| PrimeSTAR ® Max | 20 μL |
| domainA F1′ | 2 μL |
| domainA R3′ | 2 μL |
| First fragment | 0.5 μL |
| Second fragment | 0.5 μL |
| Third fragment | 0.5 μL |
| H₂O | 15.5 μL |

In an illustrated embodiment, the overlap PCR program is as follows:

after preparing the required reagents for the overlap PCR program, a PCR instrument is preheated at 98° C. for 3 min to denature DNA of the recovered product, and then an amplification cycle is performed, including: maintaining at 98° C. for 30 s to denature the recovered product; decreasing the temperature to a renaturation temperature at 50° C. and maintaining for 30 s to fully anneal designated primers (referred as to domainA F1′ and domainA R3′) and the recovered product (referred as to the first fragment, the second fragment, and the third fragment); increasing the temperature to 72° C. and maintaining for 3 s to amplify the fragments on the recovered product, thereby extending the designated primers on the recovered product and synthesizing DNA (also referred as to a synthesized plasmid); thereafter the above-mentioned cycle is repeated for 32 times to obtain an overlap PCR product, and then the temperature is maintained at 72° C. for 5 min to extend the overlap PCR product completely; and finally, the overlap PCR product is stored at 4° C.

Specially, the first fragment is the PCR product of the designated primers domainA F1′ and domainA R1′, and the gene sequence of the first fragment is SEQ ID NO: 7 as:

TGAGCGGATAACAATTCCCCTCTAGAAATAATTTTGTTTAACTTTAAGAA
GGAGATATACCAATGCACCACCACCATCACCACGTGGACGCGAAATTCGA
CGCGGATAACAACTTTAACAAAGAGCAGCAGAACGCATTCTATGAAATCC
TGAACATGCCGAACCTGAATGAAGAACAGCTGAACGGCTTCATCCAGTCT
CTGAAAGATGATCCGTCCCAGTCCGCGAACCTGCT;

the second fragment is the PCR product of the designated primers domainA F2′ and domainA R2′, and the gene sequence of the second fragment is SEQ ID NO: 8 as:

GATCCGTCCCAGTCCGCGAACCTGCTGAGCGAAGCTAAAAAACTGAACGA
AAGCCAGGCGCCGAAGCAGGCGCCGAAAGTTGATGCTAAGTTTGACGCAG
ACAATAATTTCAATAAGGAACAACAAAATGCGTTTTACGAGATTCTTAAT
ATGCCAAATCTTAACGAGGAGCAACTTAACGGTTTTATTCAAAGCCTAAA
AGACGACCCTAGTCAAAGTGCTAATTTATT;

and
the third fragment is the PCR product of the designated primers domainA F3′ and domainA R3′, and the gene sequence of the third fragment is SEQ ID NO: 9 as:

GACGACCCTAGTCAAAGTGCTAATTTATTATCTGAGGCGAAGAAGTTAAA
TGAGAGTCAAGCACCTAAACAAGCTCCTAAGGTCGACGCGAAA.

Then, 10× loading buffer is added to the overlap PCR product to conduct a 1% agarose gel electrophoresis at 150 V for 15 min to obtain another gel, thereafter observing another nucleic acid band and cutting and recovering the gel containing the target gene of the overlap PCR product.

Specially, the target gene recovered from the cutting gel after the overlap PCR is the mutated target gene.

In the step 3, the double digestion is performed on the template and the mutated target gene to obtain the first enzymatic cleavage product and the second enzymatic cleavage product, respectively. Specially, the template is a single repeat SpA11 protein expression precursor vector in domain A.

A process for the double digestion is as follows: performing the double digestion with restriction endonucleases <Xba I, Sal I> on the template at 37° C. for 2 hours (h). Specially, the Xba I is a restriction endonuclease identifying a T\hat{ }CTAGA site and the Sal I is a restriction endonuclease identifying a G\hat{ }TCGAC site.

A system for the double digestion performed on the template is as follows:

| Reagent name | Usage amount |
|---|---|
| Synthesized plasmid | 45 μL |
| Xba I | 1 μL |
| Sal I | 1 μL |
| Tango buffer | 6 μL |
| Distilled deionized water (ddH₂O) | 7 μL-60 μL |

At the same time, the double digestion with restriction endonucleases <Xba I, Sal I> is also performed on the mutated target gene obtained in the step 2 at 37° C. for 2 h.

A system for the double digestion performed on the mutated target gene is as follows:

| Reagent name | Usage amount |
|---|---|
| Recovered overlap PCR product | 38 μL |
| Xba I | 1 μL |
| Sal I | 1 μL |
| Tango buffer | 5 μL |
| ddH₂O | 5 μL-50 μL |

A product of the double digestion performed on the mutated target gene is a mixture of a first enzymatic cleavage transition product (i.e., a fragment of the mutated target gene) inserted into the gene digestion system and the gene sequence of the foregoing mixture is SEQ ID NO: 10 as follows:

CTAGAAATAATTTTGTTTAACTTTAAGAAGGAGATATACCAATGCACCAC
CACCATCACCACGTGGACGCGAAATTCGACGCGGATAACAACTTTAACAA
AGAGCAGCAGAACGCATTCTATGAAATCCTGAACATGCCGAACCTGAATG
AAGAACAGCTGAACGGCTTCATCCAGTCTCTGAAAGATGATCCGTCCCAG
TCCGCGAACCTGCTGAGCGAAGCTAAAAAACTGAACGAAAGCCAGGCGCC
GAAGCAGGCGCCGAAAGTTGATGCTAAGTTTGACGCAGACAATAATTTCA
ATAAGGAACAACAAAATGCGTTTTACGAGATTCTTAATATGCCAAATCTT
AACGAGGAGCAACTTAACGGTTTTATTCAAAGCCTAAAAGACGACCCTAG
TCAAAGTGCTAATTTATTATCTGAGGCGAAGAAGTTAAATGAGAGTCAAG
CACCTAAACAAGCTCCTAAGG.

A product of the double digestion performed on the template is a mixture of a second enzymatic cleavage transition product and a vector digestion system for the gene to be inserted after the double digestion, and the gene sequence of the foregoing mixture is SEQ ID NO: 11 as follows:

TCGACGCGAAAGGATCCGGCGGCGGCTGCTAACTCGAGGCACCACCACCA
CCACCACTGAGATCCGGCTGCTAACAAAGCCCGAAAGGAAGCTGAGTTGGCTG
CTGCCACCGCTGAGCAATAACTAGCATAACCCCTTGGGGCCTCTAAACGGGTCT
TGAGGGGTTTTTTGCTGAAAGGAGGAACTATATCCGGATTGGCGAATGGGACG
CGCCCTGTAGCGGCGCATTAAGCGCGGCGGGTGTGGTGGTTACGCGCAGCGTG
ACCGCTACACTTGCCAGCGCCCTAGCGCCCGCTCCTTTCGCTTTCTTCCCTTCCT
TTCTCGCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATTAGGG
TGATGGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAACACT
CAACCCTATCTCGGTCTATTCTTTTGATTTATAAGGGATTTTGCCGATTTCGGCC
TATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAAC
CCCTATTTGTTTATTTTTCTAAATACATTCAAATATGTATCCGCTCATGAATTAA
TTCTTAGAAAAACTCATCGAGCATCAAATGAAACTGCAATTTATTCATATCAGG
ATTATCAATACCATATTTTTGAAAAAGCCGTTTCTGTAATGAAGGAGAAAACTC
ACCGAGGCAGTTCCATAGGATGGCAAGATCCTGGTATCGGTCTGCGATTCCGA
CTCGTCCAACATCAATACAACCTATTAATTTCCCCTCGTCAAAAATAAGGTTAT
CAAGTGAGAAATCACCATGAGTGACGACTGAATCCGGTGAGAATGGCAAAAGT
TTATGCATTTCTTTCCAGACTTGTTCAACAGGCCAGCCATTACGCTCGTCATCA
AAATCACTCGCATCAACCAAACCGTTATTCATTCGTGATTGCGCCTGAGCGAGA
CGAAATACGCGATCGCTGTTAAAAGGACAATTACAAACAGGAATCGAATGCAA
CCGGCGCAGGAACACTGCCAGCGCATCAACAATATTTTCACCTGAATCAGGAT
ATTCTTCTAATACCTGGAATGCTGTTTTCCCGGGGATCGCAGTGGTGAGTAACC
ATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTA
CCTTTGCCATGTTTCAGAAACAACTCTGGCGCATCGGGCTTCCCATACAATCGA
TAGATTGTCGCACCTGATTGCCCGACATTATCGCGAGCCCATTTATACCCATAT
AAATCAGCATCCATGTTGGAATTTAATCGCGGCCTAGAGCAAGACGTTTCCCGT
TGAATATGGCTCATAACACCCCTTGTATTACTGTTTATGTAAGCAGACAGTTTT
ATTGTTCATGACCAAAATCCCTTAACGTGAGTTTTCGTTCCACTGAGCGTCAGA
CCCCGTAGAAAAGATCAAAGGATCTTCTTGAGATCCTTTTTTTCTGCGCGTAAT
CTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAG
ATACCAAATACTGTCCTTCTAGTGTAGCCGTAGTTAGGCCACCACTTCAAGAAC
TCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACC
GGATAAGGCGCAGCGGTCGGGCTGAACGGGGGGTTCGTGCACACAGCCCAGCT
TGGAGCGAACGACCTACACCGAACTGAGATACCTACAGCGTGAGCTATGAGAA
AGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTTTGTGA
TGCTCGTCAGGGGGGCGGAGCCTATGGAAAAACGCCAGCAACGCGGCCTTTTT
ACGGTTCCTGGCCTTTTGCTGGCCTTTTGCTCACATGTTCTTTCCTGCGTTATCC
CCTGATTCTGTGGATAACCGTATTACCGCCTTTGAGTGAGCTGATACCGCTCGC
CGCAGCCGAACGACCGAGCGCAGCGAGTCAGTGAGCGAGGAAGCGGAAGAGC
GCCTGATGCGGTATTTTCTCCTTACGCATCTGTGCGGTATTTCACACCGCATATA
TGGTGCACTCTCAGTACAATCTGCTCTGATGCCGCATAGTTAAGCCAGTATACA
CTCCGCTATCGCTACGTGACTGGGTCATGGCTGCGCCCCGACACCCGCCAACAC
CCGCTGACGCGCCCTGACGGGCTTGTCTGCTCCCGGCATCCGCTTACAGACAAG
CTGTGACCGTCTCCGGGAGCTGCATGTGTCAGAGGTTTTCACCGTCATCACCGA
AACGCGCGAGGCAGCTGCGGTAAAGCTCATCAGCGTGGTCGTGAAGCGATTCA
CAGATGTCTGCCTGTTCATCCGCGTCCAGCTCGTTGAGTTTCTCCAGAAGCGTT
AATGTCTGGCTTCTGATAAAGCGGGCCATGTTAAGGGCGGTTTTTTCCTGTTTG
GTCACTGATGCCTCCGTGTAAGGGGGATTTCTGTTCATGGGGGTAATGATACCG
ATGAAACGAGAGAGGATGCTCACGATACGGGTTACTGATGATGAACATGCCCG
GTTACTGGAACGTTGTGAGGGTAAACAACTGGCGGTATGGATGCGGCGGGACC
AGAGAAAAATCACTCAGGGTCAATGCCAGCGCTTCGTTAATACAGATGTAGGT
GTTCCACAGGGTAGCCAGCAGCATCCTGCGATGCAGATCCGGAACATAATGGT
GCAGGGCGCTGACTTCCGCGTTTCCAGACTTTACGAAACACGGAAACCGAAGA
CCATTCATGTTGTTGCTCAGGTCGCAGACGTTTTGCAGCAGCAGTCGCTTCACG
TTCGCTCGCGTATCGGTGATTCATTCTGCTAACCAGTAAGGCAACCCCGCCAGC
CTAGCCGGGTCCTCAACGACAGGAGCACGATCATGCGCACCCGTGGGGCCGCC
ATGCCGGCGATAATGGCCTGCTTCTCGCCGAAACGTTTGGTGGCGGGACCAGT
GACGAAGGCTTGAGCGAGGGCGTGCAAGATTCCGAATACCGCAAGCGACAGG
CCGATCATCGTCGCGCTCCAGCGAAAGCGGTCCTCGCCGAAAATGACCCAGAG
CGCTGCCGGCACCTGTCCTACGAGTTGCATGATAAAGAAGACAGTCATAAGTG
CGGCGACGATAGTCATGCCCCGCGCCCACCGGAAGGAGCTGACTGGGTTGAAG
GCTCTCAAGGGCATCGGTCGAGATCCCGGTGCCTAATGAGTGAGCTAACTTAC
ATTAATTGCGTTGCGCTCACTGCCCGCTTTCCAGTCGGGAAACCTGTCGTGCCA
GCTGCATTAATGAATCGGCCAACGCGCGGGGAGAGGCGGTTTGCGTATTGGGC
GCCAGGGTGGTTTTTCTTTTCACCAGTGAGACGGGCAACAGCTGATTGCCCTTC
ACCGCCTGGCCCTGAGAGAGTTGCAGCAAGCGGTCCACGCTGGTTTGCCCCAG
CAGGCGAAAATCCTGTTTGATGGTGGTTAACGGCGGGATATAACATGAGCTGT
CTTCGGTATCGTCGTATCCCACTACCGAGATATCCGCACCAACGCGCAGCCCGG
ACTCGGTAATGGCGCGCATTGCGCCCAGCGCCATCTGATCGTTGGCAACCAGC
ATCGCAGTGGGAACGATGCCCTCATTCAGCATTTGCATGGTTTGTTGAAAACCG
GACATGGCACTCCAGTCGCCTTCCCGTTCCGCTATCGGCTGAATTTGATTGCGA
GTGAGATATTTATGCCAGCCAGCCAGACGCAGACGCGCCGAGACAGAACTTAA
TGGGCCCGCTAACAGCGCGATTTGCTGGTGACCCAATGCGACCAGATGCTCCA
CGCCCAGTCGCGTACCGTCTTCATGGGAGAAAATAATACTGTTGATGGGTGTCT
GGTCAGAGACATCAAGAAATAACGCCGGAACATTAGTGCAGGCAGCTTCCACA
GCAATGGCATCCTGGTCATCCAGCGGATAGTTAATGATCAGCCCACTGACGCG
TTGCGCGAGAAGATTGTGCACCGCCGCTTTACAGGCTTCGACGCCGCTTCGTTC
TACCATCGACACCACCACGCTGGCACCCAGTTGATCGGCGCGAGATTTAATCG
CCGCGACAATTTGCGACGGCGCGTGCAGGGCCAGACTGGAGGTGGCAACGCCA
ATCAGCAACGACTGTTTGCCCGCCAGTTGTTGTGCCACGCGGTTGGGAATGTAA
TTCAGCTCCGCCATCGCCGCTTCCACTTTTTCCCGCGTTTTCGCAGAAACGTGGC
TGGCCTGGTTCACCACGCGGGAAACGGTCTGATAAGAGACACCGGCATACTCT
GCGACATCGTATAACGTTACTGGTTTCACATTCACCACCCTGAATTGACTCTCT
TCCGGGCGCTATCATGCCATACCGCGAAAGGTTTTGCGCCATTCGATGGTGTCC
GGGATCTCGACGCTCTCCCTTATGCGACTCCTGCATTAGGAAGCAGCCCAGTAG
TAGGTTGAGGCCGTTGAGCACCGCCGCCGCAAGGAATGGTGCATGCAAGGAGA
TGGCGCCCAACAGTCCCCCGGCCACGGGGCCTGCCACCATACCCACGCCGAAA
CAAGCGCTCATGAGCCCGAAGTGGCGAGCCCGATCTTCCCCATCGGTGATGTC
GGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCA
CTATAGGGGAATTGTGAGCGGATAACAATTCCCCT.

The first enzymatic cleavage transition product is added into 10× loading buffer to conduct a 1% agarose gel electrophoresis at 150 V to obtain a gel, and then a nucleic acid band is observed 15 min later, and the gel is cut and recovered, thereby obtaining the first enzymatic cleavage product (containing the mutated target gene). Specially, the gene sequence of the first enzymatic cleavage product with the inserted mutated target gene is the same as the gene sequence of SEQ ID NO: 10.

The second enzymatic cleavage transition product is added into 10× loading buffer to conduct a 1% agarose gel electrophoresis at 150 V to obtain another gel, and then a nucleic acid band is observed 15 min later, and the another gel is cut and recovered, thereby obtaining the second enzymatic cleavage product (i.e., a fragment of the vector). Specially, the gene sequence of the second enzymatic cleavage product (i.e., the vector) is the same as the gene sequence of SEQ ID NO: 11.

In the step 4, the T4 ligation is performed between the first enzymatic cleavage product and the second enzymatic cleavage product to obtain the ligation product, the ligation product is cultured on the LB agar plate with kanamycin to obtain the plasmid, and then the plasmid is extracted from the LB agar plate with kanamycin.

Firstly, a method for obtaining the T4 ligation between the first enzymatic cleavage product and the second enzymatic cleavage product is as follows:

a ligation system:

| Reagent name | Usage amount |
|---|---|
| pET28a <Nco I, Xho I> | 2 μL |
| Recovered PCR products <Nco I, Xho I> | 18 μL |
| Solution I | 20 μL-40 μL |

Specially, pET28a refers to a bacterial vector for expression of N-terminally 6×His-tagged proteins with a thrombin site, Nco I is a restriction enzyme identifying a C\hat{ }CATGG site, Xho I is a restriction enzyme identifying a C\hat{ }TCGAG site, and the recovered PCR products are also referred to as the first enzymatic cleavage product and the second enzymatic cleavage product. The ligation system is performed at 16° C. to achieve the T4 ligation for 5 h, thereby to obtain the ligation product.

Then, a method for culturing the ligation product on a LB agar plate with kanamycin to obtain a plasmid and then extracting the plasmid from the LB agar plate with kanamycin includes the following steps:
transferring the ligation product to DH5α competent cells, incubating the transferred DH5α competent cells on ice for 30 min, and then performing a heat shock at 42° C. for 90 s;
re-incubating the incubated DH5α competent cells on ice for 2-3 min;
adding a non-resistant LB medium to the re-incubated DH5α competent cells in a flask to perform shake-flask culture; centrifuging the flask to take a resuspended bacterial solution with the non-resistant LB medium, and smearing the resuspended bacterial solution on the LB agar plate with kanamycin; among which, an amount of the non-resistant LB medium is 500 μL, a time for the shake-flask culture is 40 min, and parameters for the centrifuging are as follows: a speed for the centrifuging of 4,000 revolutions per minute (rpm) and a time for the centrifuging of 5 min;
placing the LB agar plate with kanamycin in an incubator for cultivation, a temperature of which is 37° C. and a time for which is in a range of 6 h to 12 h; and
after a period of time, screening positive monoclonal bacterial strains P on the LB agar plate with kanamycin to perform sample sequencing to obtain sequencing results, taking a bacterial strain with a complete sequencing result based on the sequencing results, and extracting the plasmid from the bacterial strain with the complete sequencing result.

In the step 5, the above extracted plasmid is used as the another template, the remaining design primers are introduced into the mutation sites of the plasmid to repeat the step 3 and the step 4, thereby constructing the multiple vectors on the multiple mutation sites of the protein A based on the domain A.

Specially, a PCR system used for the plasmid is as follows:

| Reagent name | Usage amount |
|---|---|
| PrimeSTAR ® Max | 20 μL |
| domainA F4′ | 2 μL |
| domainA R4′ | 2 μL |
| domainA-5 plasmid | 0.5 μL |
| H₂O | 15.5 μL |

A PCR program for the extracted plasmid is as follows:

after preparing the required reagents for the PCR program, a PCR instrument is preheated at 98° C. for 3 min to denature DNA of the another template of domainA-5 plasmid (i.e., the extracted plasmid from the bacterial strain with the complete sequencing result), and then an amplification cycle is performed, including: maintaining at 98° C. for 30 s to denature the another template; decreasing the temperature to a renaturation temperature at 50° C. and maintaining for 30 s to fully anneal the remaining design primers (i.e., domainA F4′ and domainA R4′) and the another template of domainA-5 plasmid; increasing the temperature to 72° C. and maintaining for 3 s to amplify fragments of the remaining design primers, thereby extending the remaining design primers on the another template and synthesizing DNA; thereafter the above-mentioned cycle is repeated for 32 times to obtain a PCR product, and then the temperature is maintained at 72° C. for 5 min to extend the PCR product completely; and finally, the PCR product is stored at 4° C.

Specially, the gene sequence of the domainA F4′ is SEQ ID NO: 12 as AATGCACCACCACCATCACCACGTCGACGCGAAATTC; and the gene sequence of the domainA R4′ is SEQ ID NO: 13 as GAGTTAGCAGCCGCCGCCGGATCCCTTAGGAGCTTGTTT.

Then, 10× loading buffer is added to the PCR product to conduct a 1% agarose gel electrophoresis at 150 V for 15 min to obtain a gel, thereafter observing a nucleic acid band and cutting and recycling the gel (also referred as to the mutated target genes).

The recovered mutated target genes and the extracted plasmid (also referred to the vector) are respectively performed double digestion with restriction endonucleases of BamH I (a restriction enzyme, identifying a G\hat{ }GATCC site) and Sal I.

A system for the foregoing double digestion is as follows:

| Reagent name | Usage amount |
|---|---|
| Mutated target genes or | 16 μL |
| vector | |
| BamH I | 1 μL |
| Sal I | 1 μL |
| Tango buffer | 2 μL-20 μL |

A product of the double digestion performed on the mutated target genes is a first enzymatic cleavage transition product. A product of the double digestion performed on the template (also referred to the vector or the extracted plasmid) is a second enzymatic cleavage transition product.

The first enzymatic cleavage transition product is added into 10× loading buffer to conduct a 1% agarose gel electrophoresis at 150 V to obtain a gel, and then a nucleic acid band is observed 15 min later, and the gel is cut and recovered. The recovered gel is the first enzymatic cleavage product.

The second enzymatic cleavage transition product is added into 10× loading buffer to conduct a 1% agarose gel electrophoresis at 150 V to obtain a gel, and then a nucleic acid band is observed 15 min later, and the gel is cut and recovered. The recovered gel is the second enzymatic cleavage product.

Furthermore, the T4 ligation is performed between the first enzymatic cleavage product and the second enzymatic cleavage product to obtain the ligation product, the ligation product is cultured on the LB agar plate with kanamycin to obtain the plasmid, and then the plasmid is extracted from the LB agar plate with kanamycin.

Firstly, a method for obtaining the T4 ligation between the first enzymatic cleavage product and the second enzymatic cleavage product is as follows:

a ligation system:

| Reagent name | Usage amount |
|---|---|
| pET28a <Nco I, Xho I> | 2 μL |
| Recovered PCR products <Nco I, Xho I> | 18 μL |
| Solution I | 20 μL-40 μL |

The ligation system is performed at 16° C. to achieve the T4 ligation for 5 h, thereby to obtain the ligation product.

And then, a method for culturing the ligation product on a LB agar plate with kanamycin to obtain a plasmid, and then extracting the plasmid from the LB agar plate with kanamycin includes the following steps:
transferring the ligation product to DH5α competent cells, incubating the transferred DH5α competent cells on ice for 30 min, and then performing a heat shock at 42° C. for 90 s;
re-incubating the incubated DH5α competent cells on ice for 2-3 min;
adding a non-resistant LB medium to the re-incubated DH5α competent cells in a flask to perform shake-flask culture; centrifuging the flask to take a resuspended bacterial solution with the non-resistant LB medium, and smearing the resuspended bacterial solution on the LB agar plate with kanamycin; among which, an amount of the non-resistant LB medium is 500 μL, a time for the shake-flask culture is 40 min, and parameters for the centrifuging are as follows: a speed for the centrifuging of 4,000 rpm and a time for the centrifuging of 5 min;
placing the LB agar plate with kanamycin in an incubator for cultivation, a temperature of which is 37° C. and a time for which is in a range of 6 h to 12 h; and
after a period of time, screening positive monoclonal bacterial strains P on the LB agar plate with kanamycin to perform sample sequencing to obtain sequencing results, taking a bacterial strain with a complete sequencing result based on the sequencing results, and extracting the plasmid from the bacterial strain with the complete sequencing result.

Finally, the obtained plasmid is domainA-5. The plasmid is one of the multiple vectors constructed on the multiple mutation sites of the protein A based on the domain A. Furthermore, the amino acid sequence of the domainA-5 is SEQ ID NO: 14 as follows:

HHHHHHVDAKFDADNNFNKEQQNAFYEILNMPNLNEEQLNGFIQSLKDDP
SQSANLLSEAKKLNESQAPKQAPKVDAKFDADNNFNKEQQNAFYEILNMP
NLNEEQLNGFIQSLKDDPSQSANLLSEAKKLNESQAPKQAPKVDAKFDAD
NNFNKEQQNAFYEILNMPNLNEEQLNGFIQSLKDDPSQSANLLSEAKKLN
ESQAPKQAPKVDAKFDADNNFNKEQQNAFYEILNMPNLNEEQLNGFIQSL
KDDPSQSANLLSEAKKLNESQAPKQAPKGSGGGC.

Furthermore, the alkali-tolerant mutation method of the protein A further includes using the multiple vectors constructed on the multiple mutation sites of the protein A based on the domain A as still another template, introducing the remaining design primers of the multiple design primers into mutation sites of the still another template to obtain mutated target genes.

Specially, the still another template is domainA-5 and the introduced design primers are domainA-6 F′ and domainA-R4′, respectively.

Then the step 3 to step 5 are repeated on the still another template of domainA-5 to obtain the multiple vectors constructed on the multiple mutation sites of the protein A based on the domain A once more. In addition, the obtained plasmid is domainA-5-6. Furthermore, the amino acid sequence of the domainA-5-6 is SEQ ID NO: 15 as follows:

HHHHHHVDAKFDADNNFNKEQQNAFYEILNMPNLNEEQLNGFIQSLKDDP
SQSANLLSEAKKLNESQAPKQAPKVDAKFDADNNFNKEQQNAFYEILNMP
NLNEEQLNGFIQSLKDDPSQSANLLSEAKKLNESQAPKQAPKVDAKFDAD
NNFNKEQQNAFYEILNMPNLNEEQLNGFIQSLKDDPSQSANLLSEAKKLN
ESQAPKQAPKVDAKFDADNNFNKEQQNAFYEILNMPNLNEEQLNGFIQSL
KDDPSQSANLLSEAKKLNESQAPKQAPKVDAKFDADNNFNKEQQNAFYEI
LNMPNLNEEQLNGFIQSLKDDPSQSANLLSEAKKLNESQAPKQAPKVDAK
FDADNNFNKEQQNAFYEILNMPNLNEEQLNGFIQSLKDDPSQSANLLSEA
KKLNESQAPKQAPKGSGGGC.

Specially, the domainA-5 and the domainA-5-6 both belong to the vectors prepared by the disclosure, and are two of the multiple vectors.

Moreover, the expressing multiple mutant proteins by using the multiple vectors at the multiple mutation sites of the protein A, respectively, includes the following steps:
transferring the multiple vectors into multiple BL21 competent cells, respectively, to mix the multiple vectors with the multiple BL21 competent cells evenly to obtain multiple mixtures;
performing a sodium dodecyl-sulfate polyacrylamide gel electrophoresis (SDS-PAGE) on the multiple mixtures to obtain multiple expressed culture products;
transferring the multiple expressed culture products to multiple LB media, respectively, to cultivate for a period of time, then respectively adding 0.1% of isopropyl β-D-thiogalactopyranoside (IPTG) to the multiple LB media for continue cultivation for a period of time and collecting multiple expressed bacterial proteins.

In the embodiment, 1 μL of the domainA-5-6 plasmid is added to a BL21 competent cell to mix evenly to obtain a mixture, then the mixture is incubated on ice for 30 min, and then a heat shock is performed at 42° C. for 90 s, followed by adding with a 500 milliliters (mL) of non-resistant LB medium in flask to perform shake-flask culture at 37° C. for 30 min, and then the non-resistant LB medium is mixed with the domainA-5-6 plasmid evenly. The flask is centrifuged to take resuspended bacterial solution with the non-resistant LB medium, and then the resuspended bacterial solution is smeared on a LB agar plate with kanamycin to screen a positive BL21 strain. The positive BL21 strain is added into a 3 mL of LB medium, and then is incubated at 37° C. for 4-5 hours, then is added with 0.1% of IPTG to incubate at 20° C. for 6-12 hours. Furthermore, 1 mL of the positive BL21 strain is taken for treatment and is detected the protein expression of the mutated target gene by SDS-PAGE gel electrophoresis (whole cell, supernatant, precipitation).

After the positive BL21 strain expresses the target protein, the expressed protein is transferred to a 300 mL of LB medium for cultivation at 37° C. for 4-5 hours, then 0.1% IPTG is added to the LB medium for cultivation overnight at 20° C., thereby collecting the expressed bacterial protein.

The ITPG is the isopropyl β-D-1-thiogalactopyranoside.

In addition, in an embodiment, 1 μL of the domainA-5 plasmid is added to a BL21 competent cell to mix evenly to obtain a mixture, and then the mixture is incubated on ice for 30 min, and then a heat shock is performed at 42° C. for 90 s, followed by adding with a 500 mL of non-resistant LB medium in flask to perform shake-flask culture at 37° C. for 30 min, and then the non-resistant LB medium is mixed with the domainA-5 plasmid evenly.

After mixing evenly, the cultivation is performed on the non-resistant LB medium and the domainA-5 to obtain a cultivate product. The cultivate product is smeared on a LB agar plate with kanamycin to screen a positive BL21 strain. The positive BL21 strain is added into a 3 mL of LB medium, and then is incubated at 37° C. for 4-5 hours, then is added with 0.1% of IPTG to incubate at 20° C. for 6-12 hours. Furthermore, 1 mL of the positive BL21 strain is taken for treatment and is detected the protein expression of the target gene by SDS-PAGE gel electrophoresis (whole cell, supernatant, precipitation).

After the positive BL21 strain expresses the target protein, the expressed protein is transferred to a 300 mL of LB medium for cultivation at 37° C. for 4-5 hours, and then 0.1% of IPTG is added to the LB medium for cultivation overnight at 20° C., thereby collecting the expressed bacterial protein.

In an illustrated embodiment, the detecting expression results of the multiple mutant proteins to obtain detection results includes the following steps:
placing each of the multiple expressed bacterial proteins in an alkaline environment for a treatment;
performing an activity detection for each the expressed bacterial protein placed in the alkaline environment by using a double immunodiffusion test; and
obtaining data of the activity detection.

In the embodiment of the protein expression, the multiple expressed bacterial proteins are respectively treated with 1 M NaOH for 24 h, and are respectively sampled in 8 h for a first time, in 12 h for a second time, and in 24 h for a third time. The sample-taken treatment is performed for three parallel times, then the samples taken after the treatment are replaced for three times, and then the samples are concentrated to 200-400 μL, thereafter performing the protein activity detection.

The protein activity detection is performed by using the double immunodiffusion test, including the following steps: heating and dissolving the agar by using physiological saline to prepare a 1% agar plate; drilling wells on the agar plate, then adding the samples at a concentration gradient of 0.5 milligram per milliliter (mg/mL), 0.25 mg/mL, 0.125 mg/mL, 0.0625 mg/mL, 0.03125 mg/mL, 0.0156 mg/mL; and diffusing the samples at 37° C. for 48 h. If the agar plate appears a positive reaction, an antibody complex forms a precipitation line at the corresponding well. If a precipitation line appears at the well of 0.125 mg/mL or even a lower concentration, it is determined that the sample has the protein activity. The precipitation line appearing at the well with the lower concentration indicates that the sample has a stronger protein activity.

In an illustrated embodiment, the screening an optimal mutant protein of the protein A based on the detection results includes the following steps.

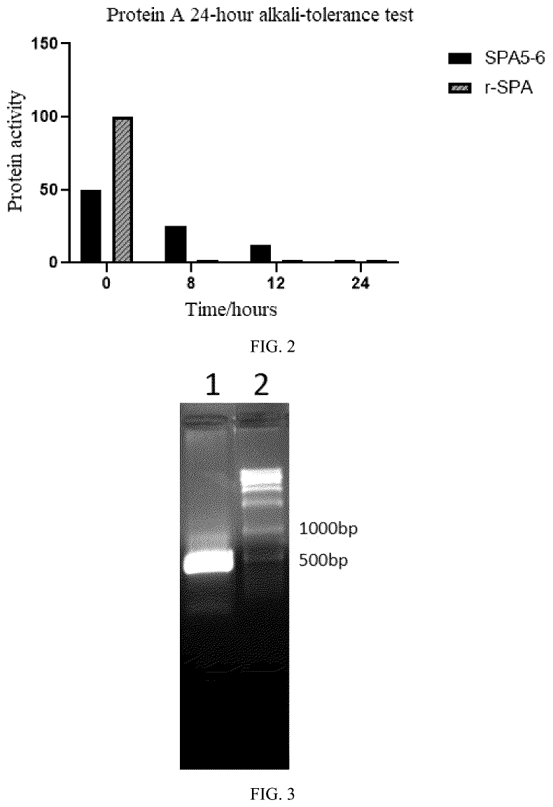

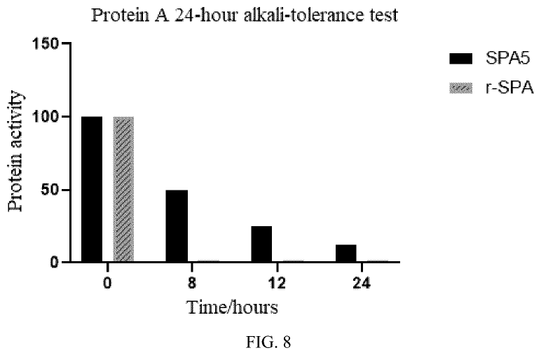

Based on the data of the protein activity detection, the alkali-tolerant mutant protein A with the highest protein activity after a preset time is screened as the optimal mutant protein A. As shown in FIG. 2 and FIG. 8. SpA5-6 no longer has the protein activity after 24 hours of treatment with 1 M NaOH, while SpA5 remains the protein activity after 24 hours of treatment with 1 M NaOH. Therefore, the SpA5 has stronger alkali tolerance compared to the SpA5-6.

Specially, the amino acid sequence of the domainA-5-6 (SEQ ID NO: 13) is as follows: HHHHHH-VDAKFD-domain A′ (1)-QAPKVDAKFD-domain A′ (2)-QAPKVDAKFD-domain A′ (3)-QAPKVDAKFD-domain A′ (4)-QAPKVDAKFD-domain A′ (5)-QAPKVDAKFD-domain A′ (6)-QAPKGSGGGC.

With reference to FIG. 2 and FIG. 8, it can be observed that the protein activities of domainA-5 and domainA-5-6 of the disclosure are much higher than that of the wild r-SpA after a period of time.

The above description only describes some illustrated embodiments of the disclosure and an explanation of the technical principles used herein. Those skilled in the related art should understand that the scope of the protection of the disclosure disclosed in the embodiments of the disclosure is not limited to the technical schemes formed by specific combinations of the aforementioned technical features, but should also cover other technical schemes formed by any combination of the aforementioned technical features or equivalent features without departing from the concept of the disclosure. For example, a technical scheme formed by replacing the technical features disclosed in (but not limited to) the embodiments of the disclosure with some technical features similar to the disclosure falls within the scope of the protection of the disclosure.

What is claimed is:

1. An alkali-tolerant mutation method of a protein A, comprising the following steps:
adding a GGGC sequence to a C-terminus of the protein A, and selecting a plurality of mutation sites of an A domain of the protein A;
constructing a plurality of vectors on the plurality of mutation sites of the protein A based on the domain A;
expressing a plurality of mutant proteins by using the plurality of vectors at the plurality of mutation sites of the protein A, respectively;
detecting expression results of the plurality of mutant proteins to obtain detection results; and
screening an optimal mutant protein of the protein A based on the detection results.

2. The alkali-tolerant mutation method of the protein A as claimed in claim 1, wherein the constructing a plurality of vectors on the plurality of mutation sites of the protein A based on the domain A comprises the following steps:
step 1, obtaining a plurality of design primers, comprising: a first design primer, a second design primer, . . . , and a n-th design primer;
step 2, introducing two of the plurality of design primers into mutation sites of a template based on a polymerase chain reaction (PCR) to obtain a mutated target gene;
step 3, performing double digestion on the template and the mutated target gene to obtain a first enzymatic cleavage product and a second enzymatic cleavage product, respectively;
step 4, performing a T4 ligation between the first enzymatic cleavage product and the second enzymatic cleavage product to obtain a ligation product, culturing the ligation product on a Lysogeny broth (LB) agar plate with kanamycin to obtain a plasmid, and then extracting the plasmid from the LB agar plate with kanamycin; and
step 5, using the plasmid as another template, introducing remaining design primers of the plurality of design primers into mutation sites of the plasmid to repeat the step 3 and the step 4, thereby constructing the plurality of vectors on the plurality of mutation sites of the protein A based on the domain A.

3. The alkali-tolerant mutation method of the protein A as claimed in claim 2, wherein the constructing a plurality of vectors on the plurality of mutation sites of the protein A based on the domain A further comprises the following steps:
step 6, using the plurality of vectors constructed on the plurality of mutation sites of the protein A based on the domain A as still another templates, introducing the remaining design primers of the plurality of design primers into mutation sites of the still another templates to obtain mutated target genes corresponding to the still another templates; and
repeating the step 3 to the step 5 with the still another templates and the mutated target genes corresponding to the still another templates to obtain a plurality of reconstructed vectors as the plurality of vectors constructed on the plurality of mutation sites of the protein A based on the domain A.

4. The alkali-tolerant mutation method of the protein A as claimed in claim 2, wherein after the performing double digestion on the template and the mutated target gene to obtain a first enzymatic cleavage product and a second enzymatic cleavage product, respectively, of the step 3, the method comprises:
adding 10× loading buffer to the first enzymatic cleavage product to conduct a 1% agarose gel electrophoresis to obtain a gel, then observing a nucleic acid band, and cutting and recycling the gel.

5. The alkali-tolerant mutation method of the protein A as claimed in claim 4, wherein the culturing the ligation product on a LB agar plate with kanamycin to obtain a plasmid, and then extracting the plasmid from the LB agar plate with kanamycin in the step 4 comprises the following steps:
transferring the ligation product to DH5α competent cells, incubating the transferred DH5α competent cells on ice, and then performing a heat shock;
re-incubating the incubated DH5α competent cells on ice;
adding a non-resistant LB medium to the re-incubated DH5α competent cells in a flask to perform shake-flask culture; centrifuging the flask to take a resuspended bacterial solution with the non-resistant LB medium, and smearing the resuspended bacterial solution on the LB agar plate with kanamycin;
placing the LB agar plate with kanamycin in an incubator for cultivation; and
after a period of time, screening positive monoclonal bacterial strains P on the LB agar plate with kanamycin to perform sample sequencing to obtain sequencing results, taking a bacterial strain with a complete sequencing result based on the sequencing results, and extracting the plasmid from the bacterial strain with the complete sequencing result.

6. The alkali-tolerant mutation method of the protein A as claimed in claim 2, wherein the expressing a plurality of mutant proteins by using the plurality of vectors at the plurality of mutation sites of the protein A, respectively, comprises the following steps:
transferring the plurality of vectors into a plurality of BL21 competent cells, respectively, to mix the plurality of vectors with the plurality of BL21 competent cells evenly to obtain a plurality of mixtures; incubating the plurality of mixtures on ice and then performing a heat shock, then re-incubating the plurality of incubated mixtures on ice; adding non-resistant LB media to the plurality of re-incubated mixtures in flasks to perform shake-flask culture; centrifuging the flasks to take resuspended bacterial solutions with the non-resistant LB media, and smearing the resuspended bacterial solutions on a plurality of LB agar plates with kanamycin; placing the plurality of LB agar plates with kanamycin in incubators for cultivation;
after a period of time, cultivating positive monoclonal bacterial strains taken from the plurality of LB agar plates with kanamycin, and then adding 0.1% isopropyl β-D-thiogalactopyranoside (IPTG) to the positive monoclonal bacterial strains, respectively, for a continue cultivation for a period of time to obtain a plurality of culture products;
performing a sodium dodecyl-sulfate polyacrylamide gel electrophoresis (SDS-PAGE) on the plurality of culture products, respectively, to obtain a plurality of expressed culture products; and
transferring the plurality of expressed culture products to a plurality of LB media, respectively, to cultivate for a period of time, then respectively adding 0.1% IPTG to the plurality of LB media for continue cultivation for a period of time and collecting a plurality of expressed bacterial proteins.

7. The alkali-tolerant mutation method of the protein A as claimed in claim 6, wherein the detecting expression results of the plurality of mutant proteins to obtain detection results comprises the following steps:
placing each of the plurality of expressed bacterial proteins in an alkaline environment for a treatment;
performing an activity detection for each of the expressed bacterial proteins placed in the alkaline environment by a double immunodiffusion test; and
obtaining data of the activity detection.

8. The alkali-tolerant mutation method of the protein A as claimed in claim 7, wherein the screening an optimal mutant protein of the protein A based on the detection results comprises:
selecting an alkali-tolerant mutant protein A with a highest activity after a preset time as the optimal mutant protein A based on the data of the activity detection.

9. The alkali-tolerant mutation method of the protein A as claimed in claim 2, wherein the plurality of design primers are domainA F1′, domainA R1′, domainA F2′, domainA R2′, domainA F3′, and domainA R3′;
wherein the gene sequence of the domainA F1′ is SEQ ID NO: 1 as TGAGCGGATAACAATTCCCCTCTAGAA;
wherein the gene sequence of the domainA R1′ is SEQ ID NO: 2 as AGCAGGTTCGCGGACTGGGACGGATC;
wherein the gene sequence of the domainA F2′ is SEQ ID NO: 3 as GATCCGTCCCAGTCCGCGAACCTGCT;
wherein the gene sequence of the domainA R2′ is SEQ ID NO: 4 as AATAAATTAGCACTTTGACTAGGGTCGTC;
wherein the gene sequence of the domainA F3′ is SEQ ID NO: 5 as GACGACCCTAGTCAAAGTGCTAATTTATT; and
wherein the gene sequence of the domainA R3′ is SEQ ID NO: 6 as CCGCCGCCGGATCCTTTCGCGTCGACCTTAGGAGCTT.
